(19) United States
(12) Patent Application Publication
(10) Pub. No.: US 2010/0292665 A1
(43) Pub. Date: Nov. 18, 2010

(54) SANITARY WATERPROOF PAD FOR TWEENS

(76) Inventors: Susan Sigel, Lurchmont, NY (US); Sari Kleinman, Larchmont, NY (US)

Correspondence Address: PATENT DEPARTMENT;LARKIN, HOFFMAN, DALY & LINDGREN, LTD., 1500 WELLS FARGO PLAZA, 7900 XERXES AVENUE SOUTH, BLOOMINGTON, MN, 55431, US

(21) Appl. No.: 12/780,752

(22) Filed: May 14, 2010

Related U.S. Application Data
(60) Provisional application No. 61/178,558, filed on May 15, 2009.

(51) Int. Cl. A61F 13/20; A61F 13/15

(52) U.S. Cl. 604/385.05; 604/385.17

(57) ABSTRACT

A sanitary pad for tween girls for use under a swimsuit. The sanitary pad includes a body having a size and shape conforming to a genital area of an average tween girl and is inconspicuous while being worn by the tween under her swimsuit. The body has a waterproof outer layer, an inner lining and a peripheral edge. The waterproof outer layer has a removable backing covering an adhesive such that upon removal of the backing, the body is removably adherably positionable in the swimsuit and remains in place during vigorous swimming activities and play, whether in or out of the water. The inner lining is capable of absorbing menstrual fluid. A vaginal plug is positioned on the inner lining so as to align comfortably at the opening of the tween's vagina.

### BACKGROUND

Many adolescent girls are uncomfortable using traditional means of feminine protection, like a tampon, when they first begin to menstruate. Accordingly, there is a need for a product designed to bridge the gap between sanitary pads and tampons in order for them to participate in activities, like swimming, that otherwise are prohibitive due to the lack of products currently available.

Traditional sanitary pads are not suitable for wearing while swimming because they absorb a lot of water, become bulky, leak, and are unsightly and uncomfortable in a swimsuit. Accordingly, there is a need for sanitary protection specifically for use by teens and pre-teens while swimming or participating in water sports and activities.

### DESCRIPTION OF THE DRAWINGS

FIG. 1 is a top plan view of a preferred embodiment of a sanitary waterproof pad intended for tween girls.

FIG. 2 is a bottom plan view of the pad of FIG. 1.

FIG. 3 is a cross-sectional view of the pad of FIG. 1 as viewed along lines 3-3.

### DESCRIPTION OF INVENTION

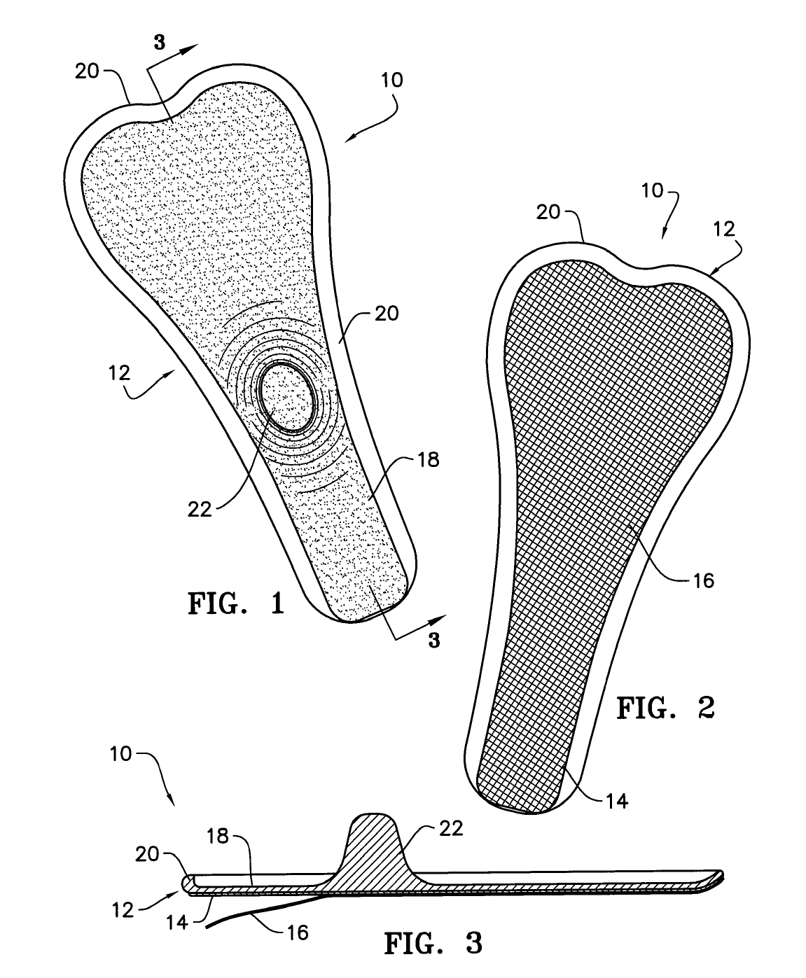

Referring now to the drawings, wherein like reference numerals designate identical or corresponding parts throughout the several views, FIG. 1 is a plan view of a preferred embodiment of a sanitary waterproof pad, indicated generally by reference numeral 10 and intended to be worn under swimsuits by preteen and young teen girls between menarche and puberty, typically between the ages of nine and fourteen (hereinafter “tweens”), who may not be accustomed, or psychologically ready, to wear a tampon, thereby providing a bridge between a traditional sanitary pad and a tampon. No insertion is necessary as the bottom of the pad 10 adheres to the swim garment, so, upon removal of the swimsuit, the wearer will effectively remove the pad as well and, like a pad, easily be able to wrap and discard it in the trash.

The pad 10 includes a body 12 having a size and shape to conform to the genital area of an average tween girl while being concealable under her swimsuit. The body 12 is preferably about three inches in length, and about two inches wide at its front end and about one inch wide at its rear end to comfortably fit between the tween's legs while still providing maximum coverage and remaining concealed under the swimsuit.

The body 12 is preferably latex and rubber free and preferably includes a flexible outer layer 14 made of waterproof material similar to waterproof bandages to prevent the absorption of water. A preferred material for the outer layer may be an elastomeric polyvinyl. The outer layer 14 preferably includes a removable or peel-off backing 16 that covers an adhesive area 17. When the backing 16 is peeled off, the exposed adhesive 17 area on the back side of the outer layer secures to the inside of the swimsuit at the desired location. The adhesive 17 is preferably waterproof so that it remains securely, yet removably positioned in place during vigorous swimming activities or when playing, whether in or out of the water.

The interior of the body 12 preferably includes a thin lining 18 made of water-resilient, yet absorbent material such as cotton and rayon to absorb any secondary flows of menstrual fluid. In the preferred embodiment, the liner preferably has a thickness similar to the inside of an adhesive bandage.

A border 20 of slightly thicker, but preferably the same material as the rest of the liner, preferably extends around the exterior periphery of the body 12. This border 20 acts as a rim to prevent menstrual leakage onto the swim garment.

Positioned near the middle of the pad 10 is a vaginal plug or mound 22 that projects a short distance above the surface of the interior lining 18 and is intended to align comfortably at the opening of the vagina. The plug 22 is preferably similar in shape to a tampon, but not as long, and is designed to conform to the opening of the vagina. The plug 22 preferably provides adequate protection comparable to approximately three-quarters the absorbency of a typical junior tampon, which are typically capable of absorbing about six grams. The plug 22 is intended to be more absorbent than the lining 18 but need not be as absorbent as conventional interlabial pads. The plug 22 is preferably made of a flexible cotton based material similar to a tampon, to provide increased comfort and protection for the young woman during vigorous swimming activities or when playing, whether in or out of the water. The preferred dimensions of the plug are about one-half-inch in length and about three-eighths inch in diameter.

It should be appreciated that the lining 18 need only be minimally absorbent because of reduced menstrual flow typically experienced while swimming due to water pressure and due to the inclusion of the vaginal plug at the opening of the vagina, which forms a physical barrier to the flow of menstrual fluid while the sanitary pad is worn.

Based on the foregoing, it should be appreciated that the pad 10 is preferably similar to a traditional sanitary pad, but is different from other products currently on the market because it is water-resilient, inconspicuous—even under a swimsuit, easy to use as no insertion is necessary, and is comfortable to wear. The pad provides adequate coverage and optimal protection while in water, where, in most cases, a young girl's period all but stops. The pad 10 can also be used as an entry into more sophisticated sanitary protection. All materials used in the pad 10 are preferably hypoallergenic.

The pad 10 is for that girl: the girl who has to sit on the ledge of the pool during P.E., but doesn't want to tell anyone why; that girl who wants to jump into the lake at camp, but can't, because she has her period and she doesn't want anyone to know; or that girl on vacation, who's on the beach blanket because she's too scared to ride the waves without adequate sanitary coverage. The pad in accordance with the present invention gives that girl the protection and piece-of-mind she needs, when she wants to swim.

1. A sanitary pad for tween girls for use under swimsuits, comprising:
a body having a size and shape conforming to a genital area of an average tween girl and which is inconspicuous while being worn by the tween under a swimsuit, the body having a waterproof outer layer, an inner lining and a peripheral edge;
the waterproof outer layer having a removable backing covering an adhesive such that upon removal of the backing, the body is removably adherably positionable in the swimsuit and remains in place during vigorous swimming activities and play, whether in or out of the water;
the inner lining comprising a material capable of absorbing menstrual fluid;
a vaginal plug positioned on the inner lining so as to align comfortably at the opening of the tween's vagina.

2. The sanitary pad of claim 1 further including a border extending around a substantial portion of the peripheral edge of the body, the border having a thickness greater than the inner lining.
